(19) United States
(12) Patent Application Publication
(10) Pub. No.: US 2026/0224597 A1
(43) Pub. Date: Aug. 6, 2026

(54) METHODS FOR ACTIVATING IMMUNE CELLS TO KILL BACTERIA

(71) Applicant: The Texas A&M University System, College Station, TX (US)

(72) Inventors: Richard H. Gomer, College Station, TX (US); Ramesh Rijal, Hattiesburg, MS (US); Ryan J. Rahman, Philadelphia, PA (US)

(73) Assignee: The Texas A&M University System, College Station, TX (US)

(21) Appl. No.: 19/151,712

(22) PCT Filed: Feb. 1, 2024
(86) PCT No.: PCT/US2024/013914

§ 371 Date: Jul. 29, 2025

Related U.S. Application Data
(60) Provisional application No. 63/482,668, filed on Feb. 1, 2023.

(51) Int. Cl. A61K 31/675; A61K 31/365; A61K 31/52; A61P 31/04

(52) U.S. Cl. CPC A61K 31/675; A61K 31/365; A61K 31/52; A61P 31/04

(57) ABSTRACT

Provided herein are methods and compositions for treating bacterial infections in a subject comprising providing to the subject an effective amount of a P2Y1 inhibitor, a PPK1 or PPK2 inhibitor, and/or IP6K inhibitor.

### RELATED APPLICATIONS

This present disclosure claims priority to U.S. Provisional Patent Application Serial No. 63/482,668, filed Feb. 1, 2023, which is incorporated by reference herein in its entirety.

### STATEMENT OF GOVERNMENT SUPPORT

This invention was made with government support under Grant No. R35 GM139486 awarded by the National Institutes of Health, National Institute of General Medical Sciences. The government has certain rights in the invention.

### FIELD OF THE INVENTION

The present invention relates generally to the field of bacteriology and medicine. More particularly, it concerns methods for treating bacterial infections by activating human immune cells, or inhibiting the ability of the bacteria to deactivate human immune cells, to kill the bacteria by treating with (i) an inhibitor of the human macrophage protein P2Y1, (ii) an inhibitor of the bacterial proteins PPK1 or PPK2 (or both), or (iii) an inhibitor of the human macrophage protein IP6K.

### BACKGROUND OF THE INVENTION

Multi-drug resistant bacterial infections are increasingly common across the world. For example, tuberculosis is one of the top twenty leading causes of death in the world (approximately 1.5 million deaths per year in 2021) and the second leading cause of death by a communicable disease in the world. Tuberculosis (TB) is caused by Mycobacterium tuberculosis bacteria (Mtb), which mostly attacks the lungs and is lethal when not treated properly. Historically, Mtb could be effectively killed by treatment with antibiotics; however, the emergence of multi-drug resistant Mtb has become a global threat, and the current standard of care is lengthy, costly, and has side effects.

In a patient with tuberculosis, the bacteria are ingested by immune system cells called macrophages. Normally, macrophages quickly kill ingested bacteria, but Mtb sends out a signal to the macrophages that causes the macrophages to not kill the Mtb, which allows the Mtb to grow and spread. Specifically, Mtb infects and replicates within the phagosomes in tissue-resident alveolar macrophages (Lawn and Zumla, 2011). In macrophages, phagosomes containing pathogens normally fuse with a lysosome to create a phagolysosome, where the pathogen is degraded by proteolytic enzymes and reactive oxygen species (Omotade and Roy, 2019; Slauch, 2011). However, Mtb inhibits the fusion of the Mtb-containing phagosome and the lysosome, and thus prevents killing of the Mtb (Russell et al., 2002; Sturgill-Koszycki et al., 1994). Mtb can be killed using drugs that mostly target bacteria (Lange et al., 2019). However, due to Mtb localization inside macrophages (Russell et al., 2002; Sturgill-Koszycki et al., 1994), the antibiotics must enter the host cells, and then enter the phagosome, and then cross the complex Mtb cell wall structure (Brennan and Nikaido, 1995) into the bacterium to effectively kill the Mtb. In addition, due to resistance developed by Mtb to currently used anti-TB drugs (Lange et al., 2019), treatment of some infections with some strains of Mtb has become difficult (Global Tuberculosis Report 2021, https://www.who.int/publications/i/item/9789240037021).

Even in the absence of multi-drug resistance, which renders standard antibiotic treatments ineffective, treatment of tuberculosis (and other bacterial infections) has serious side effects, takes a long time to treat the patient, and is expensive. Treatment costs per patient for conventional tuberculosis are approximately $20,000; treatment costs per patient for multi-drug resistant tuberculosis are approximately $182,000; and treatment costs per patient for extensively drug-resistant tuberculosis are approximately $576,000.

What is needed therefore are methods for treating bacterial infections that minimize the problems of standard treatments (including resistance, cost, and time). The present invention does so by blocking the mechanism by which bacteria evade killing by the immune system.

Human alveolar macrophages express purinergic P2Y1 receptors, and P2Y1 is highly expressed in M2 macrophages (Layhadi and Fountain, 2019), a characteristic phenotype of Mtb-infected macrophages (Huang et al., 2015). Mtb synthesizes polyphosphate (polyP), a linear polymer of inorganic polyphosphate, by polyphosphate kinase (PPK) enzymes (Singh et al., 2016). Mtb possesses PPK1 and PPK2 enzymes which are absent in humans (Singh et al., 2016). Other bacteria that possess PPK1 and/or PPK2 enzymes and can affect the respiratory system upon infection include: Pseudomonas aeruginosa, Legionella pneumophila, and Listeria monocytogenes (particularly in immunocompromised individuals).

A dual specificity inhibitor, gallein, inhibits both PPK1 and PPK2 enzyme activity in Pseudomonas aeruginosa, and attenuates the virulence of P. aeruginosa in Caenorhabditis elegans (Neville et al., 2021).

Inositol pyrophosphates are key regulators of phosphate homeostasis in various eukaryotic species (Azevedo and Saiardi, 2017; Lee et al., 2020), and the key enzymes that generate inositol pyrophosphates are inositol hexakisphosphate kinases (IP6Ks) (Lee et al., 2020; Shears, 2018). Loss of IP6K in mice reduces levels of platelet polyP (Ghosh et al., 2013).

### SUMMARY OF THE INVENTION

Provided herein are methods for treating bacterial infections by activating human immune cells to kill bacteria by treating with therapeutically effective amounts of (i) an inhibitor of the human macrophage protein P2Y1, (ii) an inhibitor of the bacterial proteins PPK1 or PPK2 (or both), or (iii) an inhibitor of the human macrophage protein IP6K.

Specifically, as disclosed herein, MRS2279, a selective high affinity competitive antagonist of the P2Y1 receptor, gallein, a polyphosphate kinase 1 and 2 inhibitor, and N6-[(4-nitrophenyl)methyl]-N2-[[3-(trifluoromethyl)phenyl]methyl]-9H-Purine-2,6-diamine (TNP), an inhibitor of inositol hexakisphosphate kinase (IP6K) and inositol 1,4,5-trisphosphate 3-kinase (IP3K), all reduce Mtb, Legionella pneumophila, and Listeria monocytogenes viability in human macrophages. These three inhibitors are thus potential therapeutics for tuberculosis and other infections caused by bacteria such as Pseudomonas aeruginosa, Legionella pneumophila, and Listeria monocytogenes where the mechanism by which the bacteria evade the immune system is to inhibit their killing in phagosomes.

In certain embodiments, the P2Y1 inhibitor is MRS2279.

In certain embodiments, the PPK1 or PPK2 inhibitor is gallein, which inhibits both PPK1 and PPK2.

In certain embodiments, the IP6K inhibitor is N6-[(4-nitrophenyl)methyl]-N2-[[3-(trifluoromethyl)phenyl]methyl]-9H-Purine-2,6-diamine (TNP).

In certain embodiments, the subject is treated with two or more of a P2Y1 inhibitor, a PPK1 or PPK2 inhibitor, or an IP6K inhibitor.

In certain embodiments, the bacterial infection being treated is caused by: Mycobacterium tuberculosis, Pseudomonas aeruginosa, Legionella pneumophila, or Listeria monocytogenes.

In certain embodiments, the subject is a human, a non-human primate, a bovine, an equine, a porcine, a canine, a feline, a guinea pig, a rat, a hamster, a rabbit, a mouse, or a mole.

### BRIEF DESCRIPTION OF THE DRAWINGS

FIG. 1 shows the chemical structure of the following P2Y1 inhibitors: MRS2179, MRS2500, BPTU, and a 4-aryl-7-hydroxylindoline derivative.

FIG. 2 shows the chemical structure of additional P2Y1 inhibitors diadenosine polyphosphate and suramin.

FIG. 3 shows the chemical structure of the P2Y1 inhibitor 1-{2-[4-chloro-1′-(2,2-dimethylpropyl)-7-hydroxy-1,2-dihydrospiro[indole-3,4′-piperidine]-1-yl]phenyl}-3-{5-chloro-[1,3]thiazolo[5,4-b]pyridine-2-yl} urea.

FIG. 4 shows the chemical structure of 2-(phenoxyaryl)-3-urea derivatives that inhibit P2Y1 activity, including: 2PXA3UD-2, 2PXA3UD-3, 2PXA3UD-9, 2PXA3UD-11, 2PXA3UD-12, and 2PXA3UD-13.

FIG. 5 shows the chemical structure of AP₄A analogs that inhibit P2Y1 activity.

FIG. 6 shows the chemical structures of additional P2Y1 inhibitors including dl-PHPB, Compound 10q, Compound 20c, Compound 4a, and Compound 7j.

FIG. 7 shows the chemical structure of MRS2279 ((1R*,2S*)-4-[2-Chloro-6-(methylamino)-9H-purin-9-yl]-2-(phosphonooxy) bicyclo[3.1.0]hexane-1-methanol dihydrogen phosphate ester diammonium salt).

FIG. 8 shows the chemical structures of: NSC35676, NSC30205, NSC345647, NSC9037, Inh1, and Inh2.

FIG. 9 shows the chemical structure of gallein (3′,4′,5′,6′-Tetrahydroxyspiro[isobenzofuran-1(3H),9′-(9H)xanthen]-3-one), a PPK1/PPK2 inhibitor.

FIG. 10 shows the chemical structure of TNP (N6-[(4-nitrophenyl)methyl]-N2-[[3-(trifluoromethyl)phenyl]methyl]-9H-Purine-2,6-diamine), an IP6K inhibitor.

FIG. 11 shows the chemical structures of additional IP6K inhibitors, including Compound 9, 20(UNC7467), SC-919, and Compound 24.

FIG. 12 shows the chemical structures of additional IP6K inhibitors that are thiadiazolidinone compounds including: LI-1753, LI-1851, LI-2355, LI-2356, and LI-2386.

FIG. 13 shows the chemical structures of additional IP6K inhibitors including: LI-2124, LI-2172, LI-2240, LI-2260, LI-2180, LI-2178, LI-2242, LI-2263, and LI-2406.

FIG. 14 shows the chemical structures of additional IP6K inhibitors including: UNC10102221, UNC10104261, UNC10105760, and UNC10225257.

FIG. 15 shows the chemical structure of an additional IP6K inhibitor, diosmetin.

FIG. 16 shows the percent cfu of viable ingested Mtb from human macrophages co-incubated with the indicated concentrations of the indicated compound (MRS2279 (16A), gallein (16B), and TNP (16C)) at 48 hours post infection. For each experiment, the average cfu/ml with no compound (0) was considered 100%. Values in FIGS. 16A-16C are mean±SEM, n=6 macrophage donors (3 males and 3 females). *indicates p<0.05, **p<0.01, ***p<0.001, and ****p<0.0001 compared to no-compound control (t-tests).

FIG. 17 shows the cfu/ml of viable ingested Mtb from human macrophages co-incubated with 1 μM of the indicated compounds (MRS 2279, gallein, and TNP) at 4 hours post infection. For each experiment, the average cfu/ml with no compound (Control) was considered 100%. Values are mean±SEM, n=6 macrophage donors (3 males and 3 females). *indicates p<0.05, **p<0.01 compared to no-compound control (t-tests).

FIG. 18 shows the metabolic activity of macrophages in the presence of the indicated concentrations of MRS2279 (18A), gallein (18B), and TNP (18C) and uninfected macrophages or Mtb infected macrophages in the presence of 1000 nM of the indicated compounds (18D). For each experiment, the metabolic activity of macrophages in the absence of compound (0) was considered 100%. (FIG. 18D) The metabolic activity of uninfected macrophages (−Mtb) or Mtb infected macrophages (+Mtb) in the presence of 1000 nM of the indicated compounds. Control indicates no added compound. Values are mean±SEM, n=6 (3 males and 3 females). *indicates p<0.05 compared to no-compound control (One-way ANOVA with Dunnett's multiple comparisons test).

FIG. 19 shows the percent cfu of viable ingested Mtb from human macrophages co-incubated with the indicated concentrations of the indicated compound at three days post infection. For each experiment, the average cfu/ml with no compound (0) was considered 100%. Values in FIG. 19A and FIG. 19B are mean±SEM, n=6 (3 males and 3 females). **p<0.01, ***p<0.001, and ****p<0.0001 compared to no-compound control (Two-way ANOVA with Dunnett's multiple comparisons test).

FIG. 20 shows the percent cfu of viable ingested Legionella pneumophila (FIG. 20A) and Listeria monocytogenes (FIG. 20B) from human macrophages co-incubated with the indicated concentrations of the indicated compound at two days post infection. For each experiment, the average cfu/ml with no compound (0) was considered 100%. Values in 20A and 20B are mean±SEM, n=6 (3 males and 3 females). *p<0.05**p<0.01, ***p<0.001, and ****p<0.0001 compared to no-compound control (Two-way ANOVA with Dunnett's multiple comparisons test).

FIG. 21 shows the percent growth of Mtb in in vitro culture co-incubated without (Control) or with 1 μg/ml INH and/or 5 μM (21A) or 50 μM (21B) gallein at the indicated days and the percent cfu of viable ingested Mtb from human macrophages co-incubated without (Control) or with 1 μg/ml INH and/or 5 μM gallein at 4 (21C) and 48 (21D) hours post infection. For each experiment, the average cfu/ml with no compound (Control) was considered 100% (21C and 21D). Values are mean±SEM of three (21A and 21B) and four (2 females and 2 males) (21C and 21D) independent experiments. *p<0.05; **p<0.01; ***p <0.001, ****p<0.0001 (Two-way ANOVA with Dunnett's multiple comparisons test for 21A and 21B, and Mann-Whitney test for FIGS. 21C and 21D).

FIG. 22 shows transmission electron microscopy images of Mtb co-incubated without or with 1 μg/ml INH and/or 5 μM gallein for 14 days (22A) and the quantification of Mtb cell envelope thickness from 22A (22B). Representative images in 22A are from at least three independent experiments. The arrows indicate the cell envelope. Bar is 100 nm in 22A. Values in 22B are mean±SEM of at least 25 cells from three independent experiments. ****p<0.0001 (One-way ANOVA with Tukey's multiple comparisons test).

### DETAILED DESCRIPTION OF THE INVENTION

Unless defined otherwise, all scientific or technical terms used herein shall have the same meaning as is commonly understood by one of skill in the art. Unless specific definitions are provided, the nomenclature used in connection with, and the procedures and techniques of organic chemistry, analytic chemistry, bacteriology, immunology, and medicinal and pharmaceutical chemistry described herein are those well-known and commonly used in the art. Standard techniques may be used for chemical synthesis, chemical analysis, pharmaceutical preparation, formulation and delivery, and treatment of subjects.

### Definitions

As used in the specification and the claims, the singular forms “a,” “an,” and “the” include the plural unless the context clearly dictates otherwise.

In the specification and the claims that follow, reference will be made to a number of terms, which shall be defined to have the following meanings:

“Administering” means providing a pharmaceutical agent or composition to a subject, and includes, but is not limited to, administering by a medical professional or self-administering.

“Administering concomitantly” means co-administering two or more agents to a subject in any manner in which the pharmacological effects of each agent are present in a subject. Concomitant administration does not require that both agents be administered in a single pharmaceutical composition, in the same dosage form, or by the same route of administration. The effects of both agents need only be overlapping for a period of time and need not be coextensive.

“Amelioration” means the lessening in severity of at least one indicator of a condition or disease. In certain embodiments, this can mean a delay or slowing in the progression of a disease. The severity of indicators may be determined by subjective or objective measures which are known to those of skill in the art.

A “control” is an alternative subject or sample used in an experiment for comparison purposes and may be either a positive or negative control.

“Decrease” can refer to any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Effective amount” of an agent refers to a sufficient amount of an agent to provide a desired effect. The amount of agent that is “effective” may vary from subject to subject, depending on many factors, such as the age, sex, weight, and general condition of the subject, the particular agent or combination of agents, and the like. It is therefore not always possible to specify a quantified “effective amount.” An appropriate “effective amount” in any subject may be determined by one of ordinary skill in the art using routine experimentation. Also, as used herein, an “effective amount” of an agent may also refer to an amount covering both therapeutically effective amounts and prophylactically effective amounts.

“Increase” can refer to any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

“Inhibit,” “inhibiting,” and “inhibition” mean to decrease an activity, response, condition, disease, or other biological parameter. This can include but is not limited to the complete ablation of the activity, response, condition, or disease as compared to the native or control level. Thus, the reduction may be a 10, 20, 30, 40, 50, 60, 70, 80, 90, 100% or any amount of reduction in between as compared to native or control levels.

“Intravenous administration” means administration into a vein.

“IP6K inhibitor” means any compound capable of inhibiting IP6K activity. Inhibition may be partial or complete inhibition.

“P2Y1 inhibitor” means any compound capable of inhibiting P2Y1 activity. Inhibition may be partial or complete inhibition.

“Parenteral administration” means administration through injection or infusion. Parenteral administration includes, but is not limited to, subcutaneous administration, intravenous administration, and intramuscular administration.

“Pharmaceutically acceptable” components may refer to components that are not biologically or otherwise undesirable, i.e., a component may be incorporated into a pharmaceutical formulation and administered to a subject as described herein without causing significant undesirable biological effects or negatively interacting with any of the other components of the formulation in which it is contained. When used in reference to administration to a human, the term means that the component has met the required standards of toxicological and manufacturing testing or that is included on the Inactive Ingredient Guide prepared by the U.S. Food and Drug Administration.

“Pharmaceutically acceptable carrier” means a carrier or excipient that is useful in preparing a pharmaceutical or therapeutic composition that is generally safe and non-toxic and includes a carrier that is acceptable for veterinary and/or human pharmaceutical or therapeutic use. Examples of a “pharmaceutically acceptable carrier” include: phosphate buffered saline solution, water, emulsions (such as an oil/water or water/oil emulsion) and/or various types of wetting agents. As used herein, the term “carrier” includes, but is not limited to, any excipient, diluent, filler, salt, buffer, stabilizer, solubilizer, lipid, or other material known in the art for use in pharmaceutical formulations.

“Pharmacologically active,” as in a “pharmacologically active” derivation or analog, can refer to a derivative or analog (e.g., a salt, ester, amide, conjugate, metabolite, isomer, fragment, etc.) having the same type of pharmacological activity as the parent compound and approximately equivalent in degree.

“PPK1 inhibitor” means any compound capable of inhibiting PPK1 activity. Inhibition may be partial or complete inhibition.

“PPK2 inhibitor” means any compound capable of inhibiting PPK2 activity. Inhibition may be partial or complete inhibition.

“PPK1 or PPK2 inhibitor” means any compound capable of inhibiting PPK1 activity or inhibiting PPK2 activity including, but not limited to a PPK1/PPK2 inhibitor. Inhibition may be partial or complete inhibition.

“PPK1/PPK2 inhibitor” means any compound capable of inhibiting both PPK1 activity and PPK2 activity. Inhibition may be partial or complete inhibition.

“Subject” means any individual who is the target of administration or treatment. The subject may be a vertebrate, for example, a mammal. In one aspect, the subject may be human, non-human primate, bovine, equine, porcine, canine, or feline. The subject may also be a guinea pig, rat, hamster, rabbit, mouse, or mole. Thus, the subject may be a human or a veterinary patient. The term “patient” refers to a subject under the treatment of a physician or veterinarian.

“Subcutaneous administration” means administration just below the skin.

“Therapeutic agent” means a pharmaceutical agent used for the cure, stabilization, amelioration, or prevention of a disease. When the term “therapeutic agent” is used or when a particular agent is specified, it is to be understood that the term includes the agent as well as pharmaceutically acceptable, pharmacologically active salts, esters, amides, proagents, conjugates, active metabolites, isomers, fragments, analogs, etc.

“Therapeutically effective” means that the amount of the composition used is of sufficient quantity to ameliorate one or more causes or symptoms of a disease or disorder. Such amelioration only requires a reduction or alteration, not necessarily elimination of the cause or symptom.

“Treating” or “treatment” means the application of one or more specific procedures used for the cure, stabilization, amelioration, or prevention of a disease. In certain embodiments, the specific procedure is the administration of one or more pharmaceutical agents.

### Overview

The invention involves treating a subject with an effective amount of one or more of a P2Y1 inhibitor, a PPK1 or PPK2 inhibitor, or an IP6K inhibitor to either ameliorate or prevent bacterial infections such as those caused by Mtb, Pseudomonas aeruginosa, Legionella pneumophila, and Listeria monocytogenes.

### P2Y1 Inhibitors

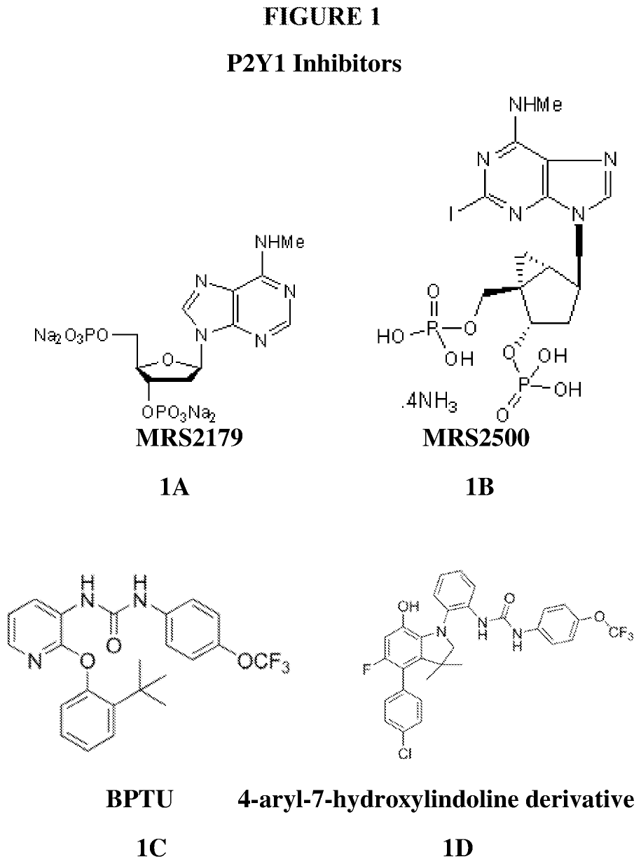

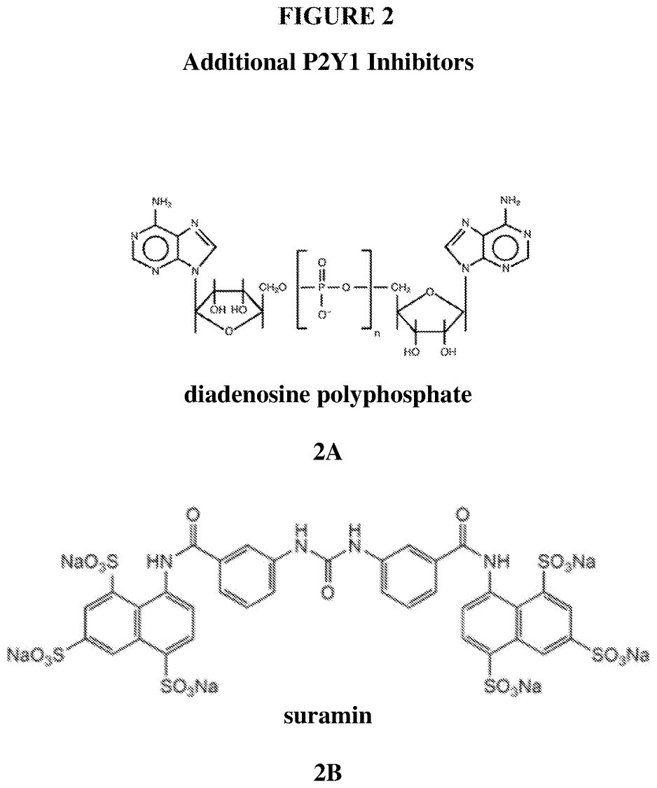

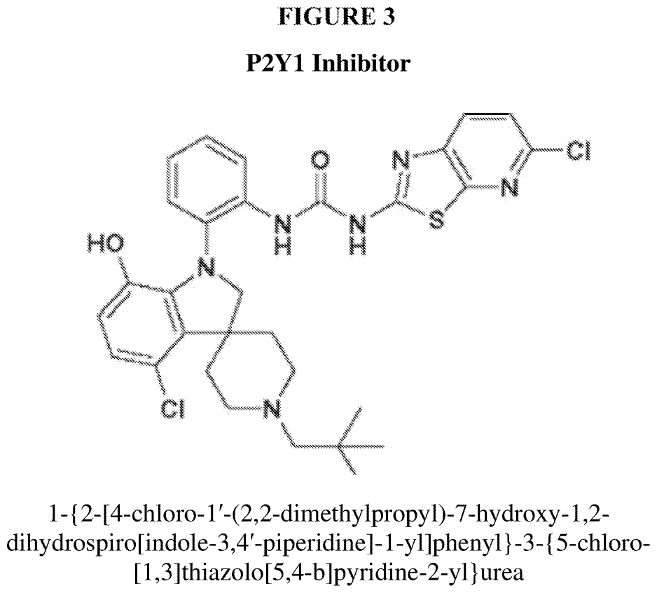

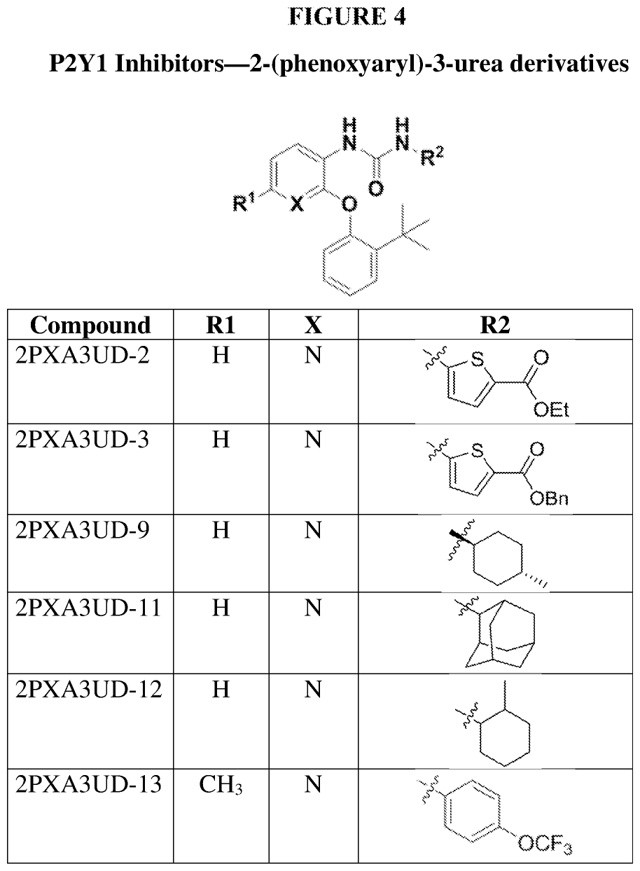

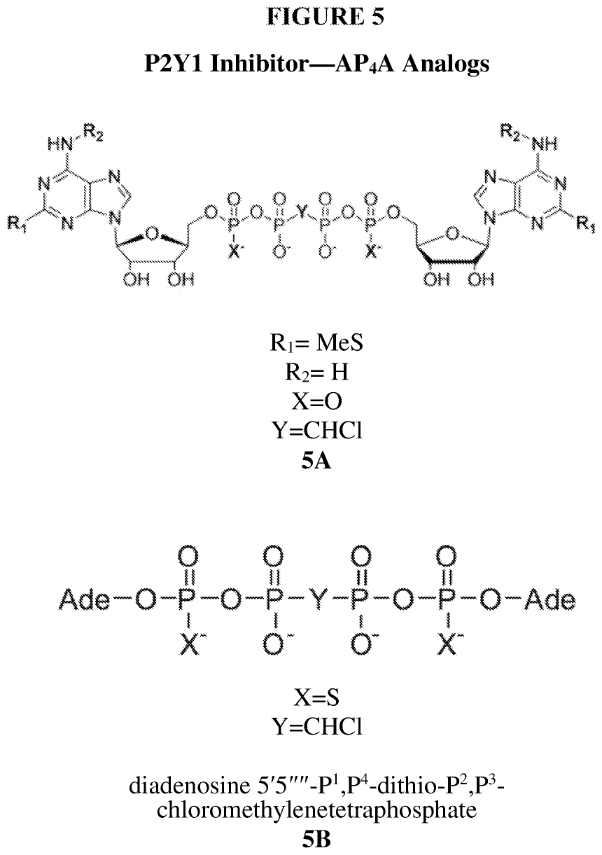

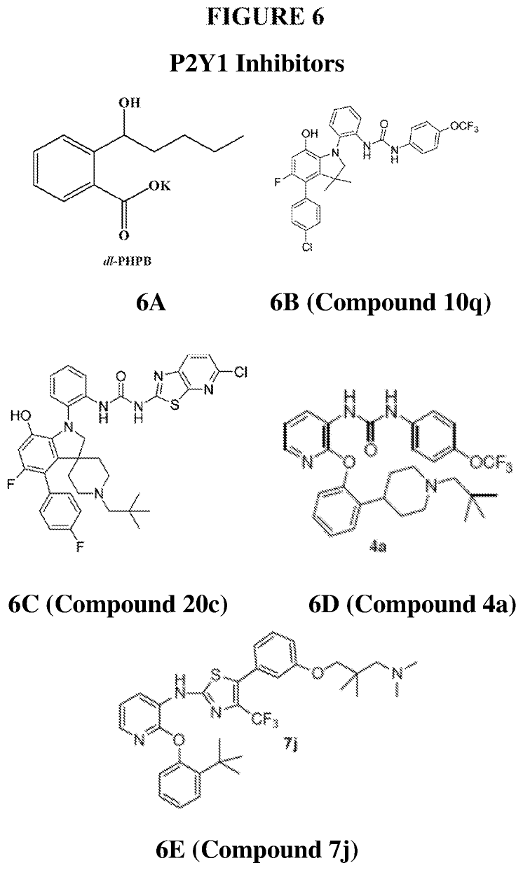

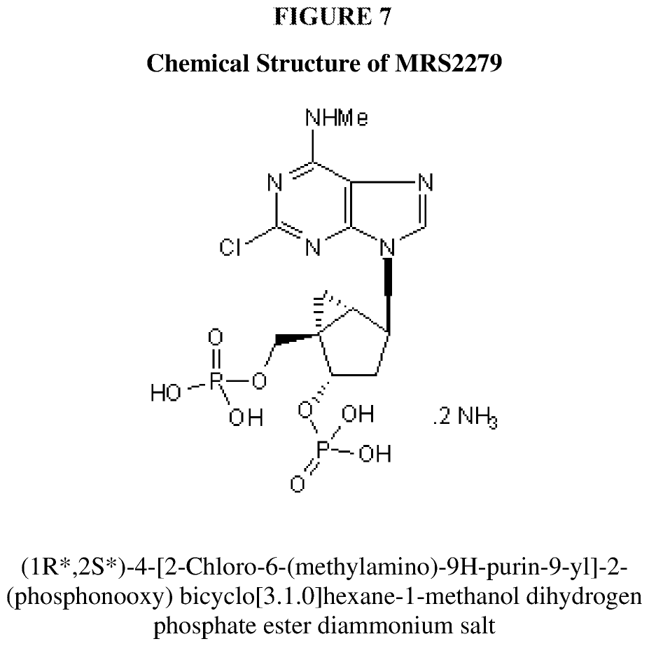

Examples of P2Y1 inhibitors useful in the invention include: MRS2179 (FIG. 1A), MRS2500((1R*,2S*)-4-[2-iodo-6-)methylamino)-9H-purin-9yl]-2-(phosphonooxy)bicyclo (3.1.0)hexane-1-methanol dihydrogen phosphate ester tetraammonium salt, FIG. 1B), BPTU (N-[2-[2-(1,1-dimethylethyl)phenoxy]-3-pyridinyl]-N′-[4-(trifluoromethoxy)phenyl] urea, FIGS. 1C), 4-aryl-7-hydroxylindoline derivative (FIG. 1D), diadenosine polyphosphate (two adenosine moieties linked through ribose 5′ carbons to a phosphate chain with the number of phosphate groups (n) from 2 to 6, FIG. 2A), suramin (FIGS. 2B), 1-{2-[4-chloro-1′-(2,2-dimethylpropyl)-7-hydroxy-1,2-dihydrospiro[indole-3,4′-piperidine]-1-yl]phenyl}-3-{5-chloro-[1,3]thiazolo[5,4-b]pyridine-2-yl} urea (FIG. 3), other 2-(phenoxyaryl)-3-urea derivatives (specifically, those illustrated in FIG. 4), Cc-Lec (see Samah 2017, which is herein incorporated by reference for its disclosures regarding Cc-Lec, which is isolated from Cerastes cerastes venom), AP₄A analogs, including preferably as illustrated in FIG. 5A and diadenosine 5′5″″-P¹,P⁴-dithio-P², P³-chloromethylenetetraphosphate (FIG. 5B), potassium 2-(1-hydroxypentyl)-benzoate (dl-PHPB, FIG. 6A), EL2Ab (see Karim 2015, which is herein incorporated by reference for its disclosures regarding EL2Ab), 4-aryl-7-hydroxyindoline-based P2Y1 antagonists (including specifically Compounds 10q and 20c (BMS-884775) as reported in Yang 2014, which structures are shown in FIGS. 6B and 6C, respectively), Compound 4a (as reported in Ruel 2013, which is herein incorporated by reference for its disclosures regarding compound 4a, FIG. 6D), Compound 7j (as reported in Pi 2013, which is herein incorporated by reference for its disclosures regarding compound 7j, FIG. 6E), and A3P5PS (adenosine-3′-phosphate-5′-phosphosulfate) (see Boyer 1996). Preferably, the P2Y1 inhibitor used in the invention is MRS2279 ((1R*,2S*)-4-[2-Chloro-6-(methylamino)-9H-purin-9-yl]-2-(phosphonooxy)bicyclo[3.1.0]hexane-1-methanol dihydrogen phosphate ester diammonium salt), which has the structure shown in FIG. 7 and inhibits the activity of P2Y1.

### PPK1 or PPK2 Inhibitors

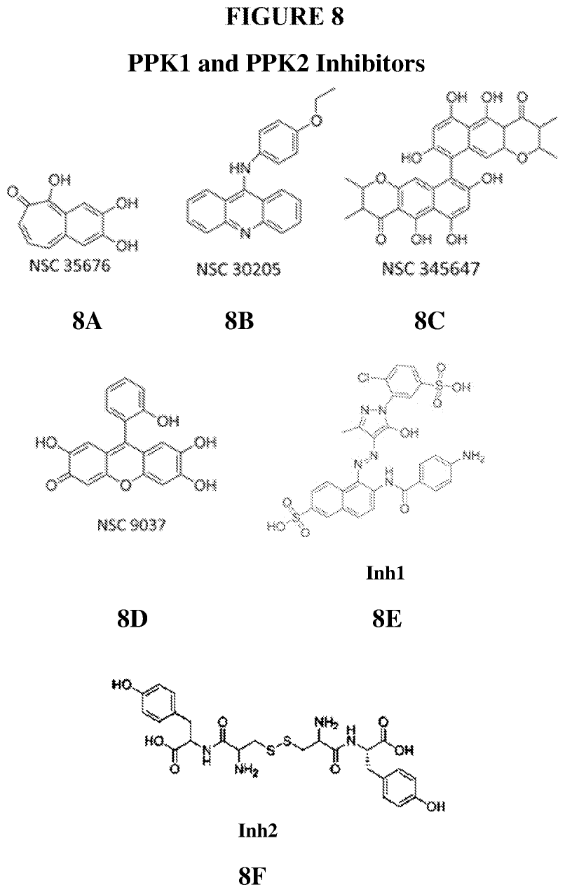

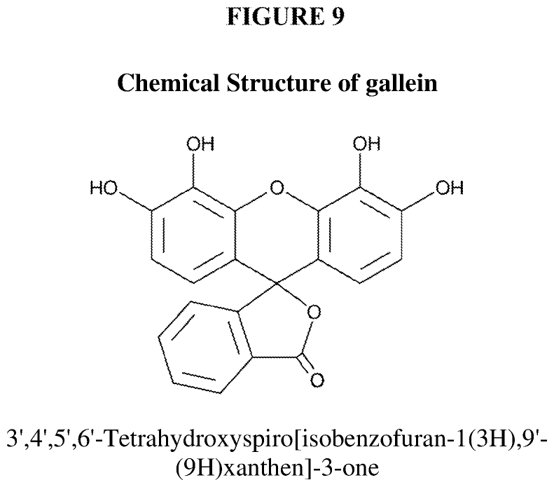

Polyphosphate kinase 1 (PPK1) and polyphosphate kinase 2 (PPK2) are bacterial proteins not present in humans. Examples of PPK1 inhibitors, PPK2 inhibitors, and PPK1/PPK2 inhibitors useful in the invention include: NSC35676 (FIG. 8A), NSC30205 (FIG. 8B), NSC345647 (FIG. 8C), NSC9037 (FIG. 8D), Inh1 (NCI Code 75963, FIG. 8E), and Inh2 (NCI Code 333714, FIG. 8F). Preferably, the PPK1/PPK2 inhibitor used in the invention is gallein (3′,4′,5′,6′-Tetrahydroxyspiro[isobenzofuran-1(3H),9′-(9H)xanthen]-3-one), which has the structure shown in FIG. 9 and inhibits the activity of both PPK1 and PPK2.

### IP6K Inhibitors

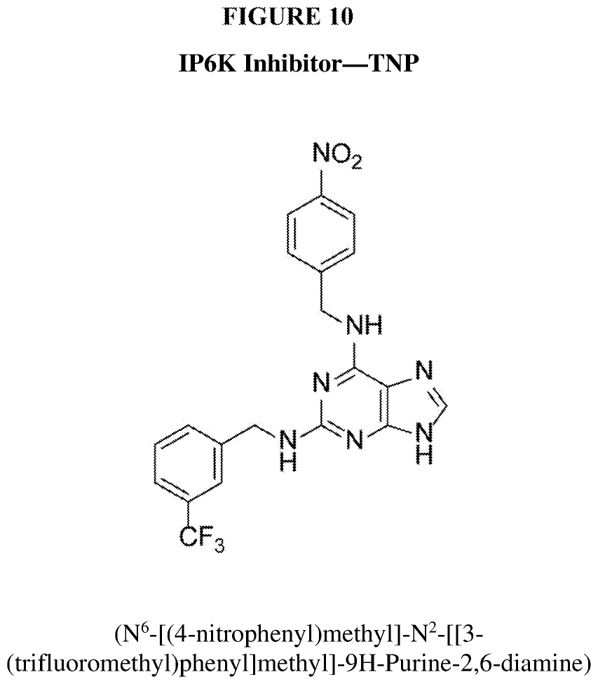

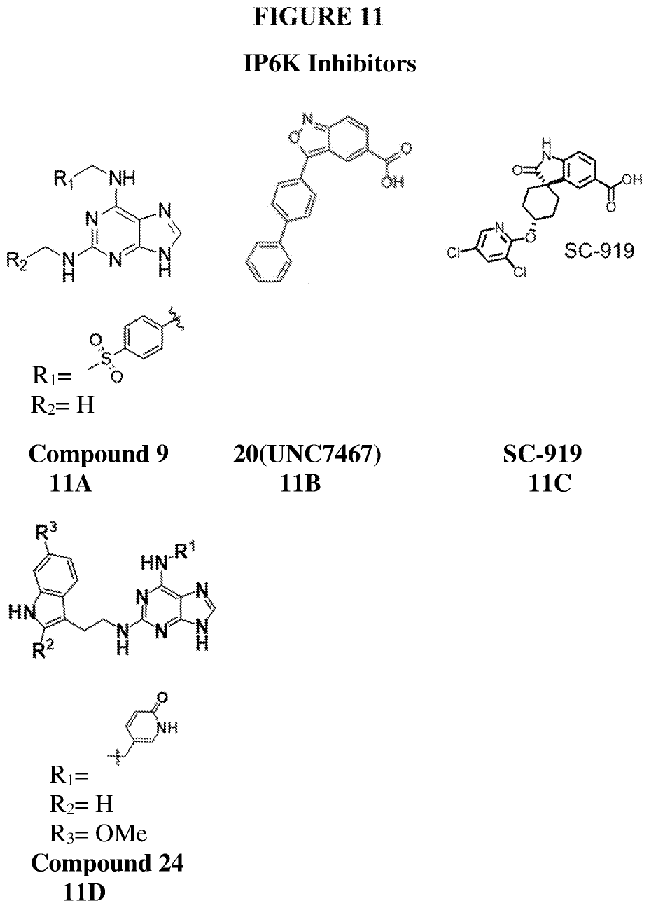

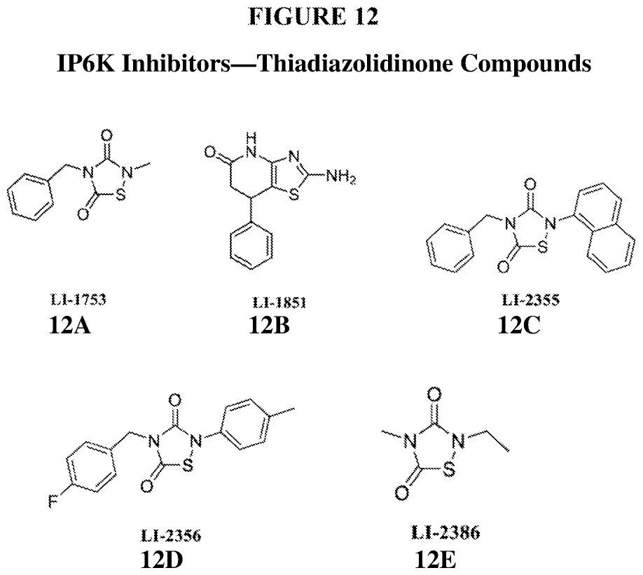

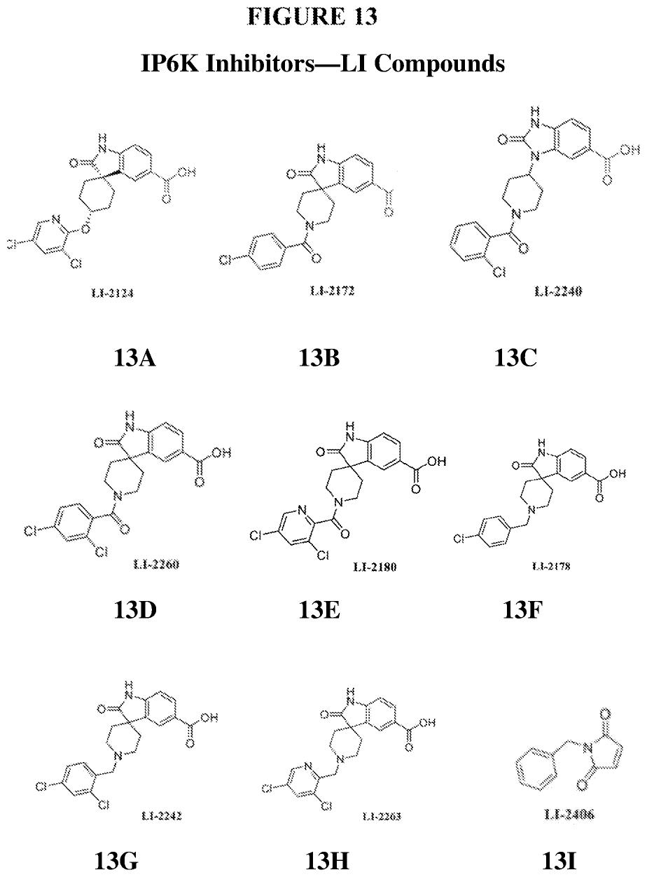

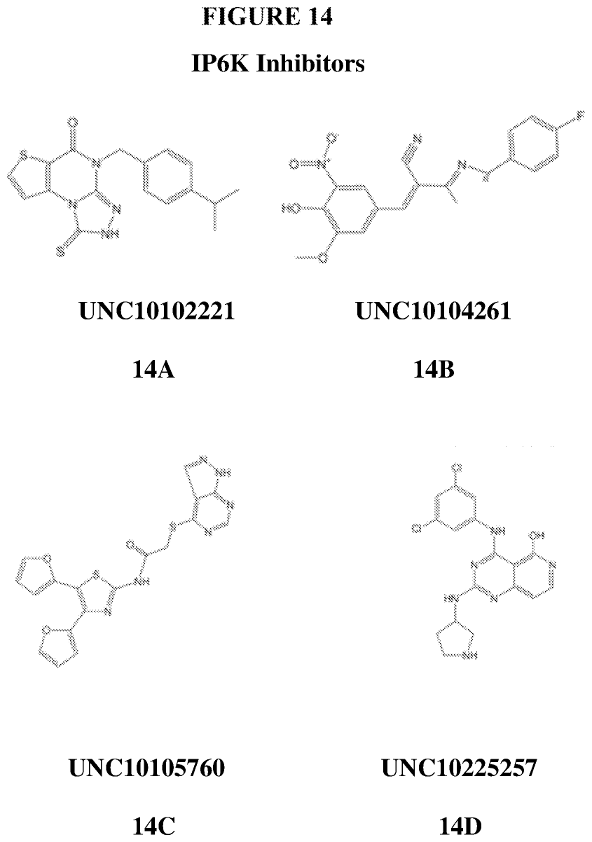

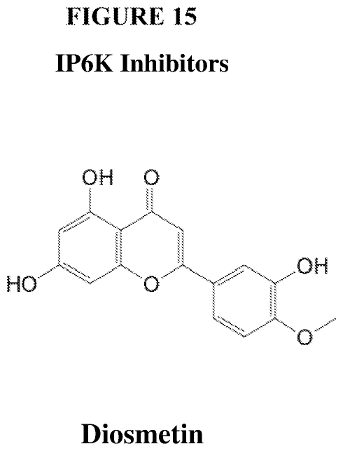

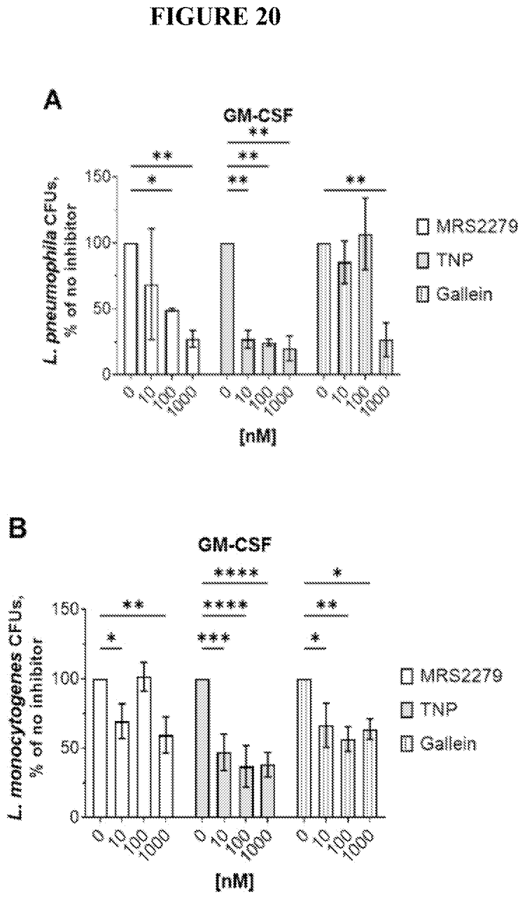

IP6K refers to human inositol hexakisphosphate kinases, which are human macrophage proteins. IP6K inhibitors reduce the activity of IP6K. IP6K inhibitors useful in the invention include: TNP (N6-[(4-nitrophenyl)methyl]-N2-[[3-(trifluoromethyl)phenyl]methyl]-9H-Purine-2,6-diamine, FIG. 10) and its analogs (see Lee 2020, which is herein incorporated by reference for its discussion regarding TNP analogs that inhibit IP6K, including but not limited to compound 9 (FIGS. 11A), 20 (UNC7467) (FIG. 11B, see Zhou 2022, which is herein incorporated by reference for its discussion regarding 20(UNC7467), which had an IC₅₀ of 8.9±1.5 nM), SC-919 (FIG. 11C, see Moritoh 2021, which is herein incorporated by reference for its discussion of SC-919), Compound 24 (FIG. 11D, see Wormald 2019, which is herein incorporated by reference for its discussion regarding Compound 24, which has an IC₅₀ of 6.13±0.08), LY 294002 (see Rajasekaran 2018, which is herein incorporated by reference for its discussions regarding LY 294002, PAO, and U73122), PAO (phenylarsine oxide), U73122 (1-[6-((17β-3-Methoxyestra-1,3,5(10)-trien-17-yl)amino)hexyl]-1H-pyrrole-2,5-dione). Additional IP6K inhibitors include LI compounds disclosed in Liao 2021, which is herein incorporated by reference for discussion of the following LI compounds (IC₅₀ values as to IP6K1): thiadiazolidinones such as LI-1753 (FIG. 12A, also known as TDZD-8, IC₅₀=2150 nM), LI-1851 (FIG. 12B, IC₅₀=1500 nM), LI-2355 (FIG. 12C, IC₅₀=10110 nM), LI-2356 (FIG. 12D, IC₅₀=9290 nM), LI-2386 (FIG. 12E, IC₅₀=4880 nM), LI-2124 (FIG. 13A, IC₅₀=2.5 nM), LI-2172 (FIG. 13B, IC₅₀=20 nM), LI-2240 (FIG. 13C, IC₅₀=1100 nM), LI-2260 (FIG. 13D, IC₅₀=33 nM), LI-2180 (FIG. 13E, IC₅₀=57 nM), LI-2178 (FIG. 13F, IC₅₀=800 nM), LI-2242 (FIG. 13G, IC₅₀=31 nM), LI-2263 (FIG. 13H, IC₅₀=220 nM), LI-2406 (FIG. 13I, IC₅₀=3560 nM). Additional IP6K inhibitors suitable for use in the disclosed methods include the following, which are disclosed in Puhl-Rubio 2018, which is herein incorporated by reference with respect to the indicated compounds (IC₅₀ values reported for IP6K2): UNC10102221 (FIG. 14A, IC₅₀=0.93 μM), UNC10104261 (FIG. 14B, IC₅₀=1.1 μM), UNC10105760 (FIG. 14C, IC₅₀=0.84 μM), and UNC10225257 (FIG. 14D, IC₅₀=1.48 μM). An additional IP6K inhibitor useful in the invention includes the flavonoid diosmetin (FIG. 15, see Gu 2019, which is herein incorporated by reference with respect to its disclosures regarding diosmetin). WO 2018/192051A1 to Terao et al. and WO 2022/125524A1 to Ernst et al., which are each incorporated by reference herein, further disclose additional IP6K inhibitors that would be suitable for the invention.

Preferably, the IP6K inhibitor used in the invention is TNP.

IP6K inhibitors do not need to directly interact with IP6K. For example, LY 294002has been shown to decrease IP6K activity through its interaction with PI3K, PAO decreases IP6K activity through its interaction with PI4K, and U73122 decreases IP6K activity through its interaction with PLC. PI3K, PI4K, and PLC are all known to be part of the same pathway as IP6K.

### Method of Treatment

For a subject, treatment pursuant to the invention involves administration of an effective amount of one or more of a P2Y1 inhibitor, a PPK1 or PPK2 inhibitor, or an IP6K inhibitor. Such administration may include oral administration or parenteral administration, which includes, but is not limited to, intravenous administration, subcutaneous administration, or intramuscular administration. A pharmaceutical formulation of an effective amount of one or more of the inhibitors described herein may include pharmaceutically acceptable carriers, including as is known in the art for the chosen method of administration. Treatment of a subject includes treatment with a therapeutically effective amount or treatment with a prophylactically effective amount of one or more of a P2Y1 inhibitor, a PPK1 or PPK2 inhibitor, or an IP6K inhibitor.

If two or more of a P2Y1 inhibitor, a PPK1 or PPK2 inhibitor, or an IP6K inhibitor are administered to a subject, such administration may occur separately or concomitantly and by the same or different routes of administration. Further, when two or more inhibitors are used for treatment, the inhibitors may be included in the same or different pharmaceutical formulations.

### EXAMPLES

The following examples demonstrate that P2Y1 inhibitors, PPK1 or PPK2 inhibitors, and IP6K inhibitors are useful to cause immune cells to kill bacteria.

### Example 1. Effect of Inhibitor Treatment on bacterial survival in human macrophages

MRS2279((1R*,2S*)-4-[2-Chloro-6-(methylamino)-9H-purin-9-yl]-2-(phosphonooxy)bicyclo[3.1.0]hexane-1-methanol dihydrogen phosphate ester diammonium salt) (Cat #2158), gallein (3′,4′,5′,6′-Tetrahydroxyspiro[isobenzofuran-1(3H),9′-(9H)xanthen]-3-one) (Cat #3090), and TNP (N6-[(4-nitrophenyl)methyl]-N2-[[3-(trifluoromethyl)phenyl]methyl]-9H-Purine-2,6-diamine) (Cat #3946) were purchased from Tocris (Minneapolis, MN), and Isonicotinic acid hydrazide (INH) (Cat #13377) was purchased from Sigma (Livonia, MI). 10 mM stocks of MRS2279 were prepared in water, 10 mM stocks of gallein and TNP were prepared in DMSO, 50 mg/ml stocks of INH were prepared in water according to the manufacturer's instructions, and aliquots of 50 μl were stored at −20° C. for further use.

### Cell Culture

Human peripheral blood was collected from healthy volunteer who gave written consent, and with specific approval from the Texas A&M University human subjects institutional review board. Peripheral blood mononuclear cells (PBMCs) were purified as previously described (Pilling et al., 2009, which is herein incorporated by reference with respect to the process for purifying PBMCs). The PBMCs were cultured in RBCSG (Roswell Park Memorial Institute Medium (RPMI) (Lonza, Walkersville, MD) containing 10% bovine calf serum (VWR Life Science Seradigm, Radnor, PA) and 2 mM 1-glutamine (Lonza)), and where indicated containing 25 ng/ml human granulocyte-macrophage colony-stimulating factor (GM-CSF) or 25 ng/ml macrophage colony-stimulating factor (M-CSF) (Biolegend, San Diego, CA) at 37° C. in a humidified chamber with 5% CO₂ in type 353219, 96-well, black/clear, tissue-culture-treated, glass-bottom plates (Corning, Big Flats, NY) with 10⁵ cells per well in 100 μL or type 353072, 96-well, tissue-culture-treated, polystyrene plates (Corning) with 10⁵ cells per well in 100 μL. At day 7, loosely adhered cells were removed by gentle pipetting, and fresh RBCSG containing GM-CSF or M-CSF (as described above) was added to the cells to a final volume of 100 μL per well, and Mtb, L. pneumophila, or L. monocytogenes survival assays were performed as described below.

The attenuated (mc-ΔleuDΔpanCD) Biosafety Level-2 strain of Mtb (a derivative of the H37Rv strain) (Sampson et al., 2004) (a gift from Dr. Jim Sacchettini, Department of Biochemistry and Biophysics, Texas A&M University, College Station, TX) was grown as described (Rock et al., 2017) in Middlebrook 7H9 broth (BD, Sparks, MD) in a type 89039-656 50 ml conical tube (Falcon, VWR Life Science Seradigm) or 7H10 agar (BD) on a type 25384-302 petri dish (VWR) at 37° C. in a humidified incubator. Both media contained 0.5% glycerol (VWR), 0.05% Tween 80 (MP Biomedicals, Solon, OH) and the Middlebrook Oleic ADC Enrichment (BD). Mtb ΔleuDΔpanCD cultures (both liquid and agar plates) were additionally supplemented with 50 μg/mL leucine (VWR Life Science Seradigm) and 50 μg/mL pantothenate (Beantown Chemical, Hudson, NH). Liquid cultures were incubated in 50 ml conical tubes on a STR200-V variable angle tube rotator (Southwest Science, Roebling, NJ) for 1 to 2 weeks until the cell density reached log-phase, and the agar plates were wrapped in plastic film to prevent desiccation and incubated for 3 to 4 weeks at 37° C. in a humidified incubator. The Biosafety Level-2 strain of L. pneumophila (Legionella pneumophila subsp. pneumophila Brenner et al. (American Type Culture Collection (ATCC) 33153)) was grown as described by ATCC (https://www.atcc.org/products/33153) in a liquid 1099 CYE Buffered Medium in a type 89039-656 50 ml conical tube (VWR) or a solid 1099 CYE Buffered Medium on a type 25384-302 petri dish (VWR) at 37° C. in a humidified incubator with 5% CO₂ for 3 days. The Biosafety Level-2 strain of L. monocytogenes (Listeria monocytogenes (Murray et al.) Pirie))(ATCC 19111) was grown as described by ATCC (https://www.atcc.org/products/19111) in a 44 Brain Heart Infusion Broth in a type 89039-656 50 ml conical tube (VWR) or a 44 Brain Heart Infusion Agar on a type 25384-302 petri dish (VWR) at 37° C. in a humidified incubator for 2 days.

### Bacterial Survival Assay

To determine the effect of MRS2279, gallein, or TNP on the survival of Mtb in human macrophages, human macrophages (from blood monocytes cultured with GM-CSF or M-CSF for 6 days) were infected with Mtb as previously described (Rijal et al., 2020), in the absence or in the presence of the inhibitor. Briefly, at day 6 after removing loosely adhered cells as described above, 100 μL RBCSG (for L. pneumophila and L. monocytogenes survival assay) or RBCSGLP (RBCSG containing 50 μg/mL leucine and 50 μg/mL pantothenate for Mtb survival in the presence or absence of INH with GM-CSF), or RBCSGLP (RBCSG containing 50 μg/mL leucine and 50 μg/mL pantothenate for the Mtb survival assay containing the indicated concentrations of the inhibitor with GM-CSF or M-CSF), were added to macrophages in each well in type 353072, 96-well, tissue-culture-treated, polystyrene plates (Corning) and incubated for 30 minutes at 37° C. Meanwhile, 1 mL of Mtb, L. pneumophila, or L. monocytogenes from a log phase culture was washed twice with RBCSG (for L. pneumophila and L. monocytogenes) or RBCSGLP (for Mtb) without GM-CSF or M-CSF by centrifugation at 12,000× g for 2 minutes in a microcentrifuge tube, resuspended in 1 mL of RBCSG (for L. pneumophila and L. monocytogenes) or RBCSGLP (for Mtb), and the optical density of 100 μl of the culture in a well in type 353072, 96-well, tissue-culture-treated, polystyrene plates (Corning) at 600 nM was measured with a Synergy Mx monochromator microplate reader (BioTek, Winooski, VT). 100 μl of RBCSG (for L. pneumophila and L. monocytogenes) or RBCSGLP (for Mtb) was used a blank. The bacteria were diluted to an optical density of 0.5 (~0.33×10⁷ L. pneumophila/ml; ~0.766×10⁷ L. monocytogenes/ml; ~10⁷ Mtb/mL) in RBCSG (for L. pneumophila and L. monocytogenes) or RBCSGLP (for Mtb). Mtb (~1 μl), L. pneumophila (~3.3 μl), or L. monocytogenes (~1.3 μl) was added to macrophages in each well such that there were ~5 bacteria per macrophage considering ~20% of the blood monocytes converted to the macrophages in the presence of GM-CSF or MCSF (Cui et al. 2021). The bacteria-macrophage co-culture plate was spun down at 500× g for 3 minutes with a Multifuge X1R Refrigerated Centrifuge (Thermo Scientific, Waltham, MA) to synchronize phagocytosis of bacteria, and incubated for 2 hours at 37° C. The supernatant medium was removed by gentle pipetting and was discarded. 100 μL of PBS warmed to 37° C. was added to the co-culture in each well, cells were gently washed to remove un-ingested extracellular bacteria, the PBS was removed, and 100 μL of RBCSG (for L. pneumophila and L. monocytogenes) or RBCSGLP (for Mtb) with MCSF or GMCSF containing 200 μg/mL gentamicin (Sigma, St. Louis, MO) was added to the cells to kill the remaining uningested bacteria or RBCSGLP (for Mtb survival in the presence or absence of INH assay) with GMCSF in the absence or in the presence of 1 μg/ml INH and/or 5 μM gallein was then added to the cells. After 2 hours, cells were washed twice with PBS as above to remove gentamicin and uningested dead bacteria. RBCSG (for L. pneumophila and L. monocytogenes) or RBCSGLP (for Mtb) (100 μL) with MCSF or GMCSF was then added to the cells. After 4 and/or 48 hours of infection, macrophages were washed as above with PBS, the PBS was removed, and cells were lysed using 200 μL 0.1% Triton X-100 (Alfa Aesar) in PBS for 5 minutes at room temperature by gentle pipetting, and 20 μl and 100 μL of the lysates were plated onto agar plates (as described above for culture of each bacterial strain). The Mtb containing agar plates were incubated for 3 to 4 weeks or until the Mtb colonies appeared, whereas L. pneumophila containing agar plates were incubated for 3 days (as described above for L. pneumophila culture) and L. monocytogenes containing agar plates were incubated for 2 days (as described for L. monocytogenes culture). Bacterial colonies obtained from plating 20 μl and 100 μl lysates were manually counted, the number of viable ingested bacterial colonies per 20 μl and 100 μl lysates was calculated and the number of viable ingested bacteria colony forming units (cfu) per ml of lysate was then calculated, which correspond to the number of viable ingested bacteria in ~2×10⁵ macrophages. To calculate percent of control, cfu/ml of the control was considered 100%. To determine the effect of the inhibitors on the survival of Mtb in human macrophages for more than 48 hours, macrophages were infected with Mtb as described above, in the absence or in the presence of the indicated concentrations of the inhibitor. The indicated concentrations of the inhibitor were then additionally added to the cells at 24 and 48 hours after Mtb infection. At 72 hours (3 days of infection), macrophages were lysed and plated onto agar (as described above for lysates). The agar plates were incubated for 3 to 4 weeks or until the Mtb colonies appeared. Mtb colonies obtained from plating 20 μl and 100 μl lysates were manually counted, the number of viable ingested Mtb colonies per 20 μl and 100 μl lysates was calculated and the number of viable ingested Mtb colony forming units (cfu) per ml of lysate was then calculated, which correspond to the number of viable ingested Mtb in ~2×10⁵ macrophages. To calculate percent of control, cfu/ml of the control was considered 100%.

### Mtb Growth Assay

To investigate the impact of gallein and/or INH on Mtb growth, Mtb from a log phase culture were washed twice with 10 ml 7H9 broth by centrifugation at 4000× g for 10 minutes in a type 89039-664 15 ml conical tube (Falcon, VWR), and resuspended in 1 mL of 7H9 broth. The optical density of 100 μl of the culture in a well of a type 353072, 96-well, tissue-culture-treated, polystyrene plate (Corning) was measured at 600 nM with a Synergy Mx monochromator microplate reader (BioTek, Winooski, VT). 100 μl of 7H9 broth was used as a blank. The bacteria were diluted to an optical density of 0.01 in 5 ml 7H9 broth in each well of a type 353046, 6 well, tissue culture-treated plate (Corning). Mtb was incubated with gallein at concentrations of 5 or 50 μM and/or 1 μg/ml INH. A 50 mM gallein stock in DMSO (VWR) was diluted to 5 mM in 7H9 broth and further serially diluted in 7H9 broth to obtain lower concentrations. The control well contained 7H9 broth with DMSO, which was similarly serially diluted in 7H9 broth, as was done for gallein. The plates were subsequently incubated in a container with humidity provided by wet paper towels at 37° C. in a humidified incubator. The optical density of 100 μl of the culture in a well in type 353072, 96-well, tissue-culture-treated, polystyrene plates (Corning) was measured daily for 14 days, and 100 μl of 7H9 broth was used a blank. The Mtb growth curves were generated as a percentage of the optical density on day 0.

### Transmission Electron Microscopy (TEM)

To investigate the effect of gallein and/or INH on Mtb cell envelope thickening, Mtb cells were prepared as described for the Mtb growth assay above. At day 14 of the growth assay, 100 μl of cells were fixed by adding an equal volume of 2× fixative, which contained 84 mM NaH₂PO₄, 68 mM NaOH, 4% paraformaldehyde (Cat #19210, Electron Microscopy Sciences), and 1% glutaraldehyde (Cat #0875, VWR). The samples were gently rocked for 1 hour and then stored at 4° C. Sample preparation for TEM imaging was performed by the Texas A&M University Microscopy and Imaging Center Core Facility's staff (RRID: SCR_022128). Briefly, on the following day, the fixed samples were collected by centrifugation for 5 minutes at 14,000× g and were postfixed and stained for 2 hours with 1% osmium tetroxide in 0.05 M HEPES at pH 7.4. The samples were then collected by centrifugation and washed with water five times, and dehydrated with acetone according to the following protocol: 15 minutes in 30%, 50%, 70%, and 90% acetone each, followed by three changes of 100% acetone, each lasting 30 minutes. During the final wash step, a minimal amount of acetone was retained, just enough to cover the pellets, to prevent rehydration of the samples. Subsequently, the samples were infiltrated with modified Spurr's resin (Quetol ERL 4221 resin; Electron Microscopy Sciences; RT 14300) in a Pelco Biowave processor (Ted Pella, Inc., Redding, CA). The process included 1:1 acetone-resin for 10 minutes at 200 W (no vacuum), 1:1 acetone-resin for 5 minutes at 200 W (vacuum at 20 inches Hg, with vacuum cycles involving open sample container caps), and 1:2 acetone-resin for 5 minutes at 200 W (vacuum at 20 inches Hg). This was followed by four cycles of 100% resin for 5 minutes each at 200 W (vacuum at 20 inches Hg). The resin was then removed, and the sample fragments were transferred to BEEM conical-tip capsules that were prefilled with a small amount of fresh resin. More resin was added to fill the capsules, and they were left to stand upright for 30 minutes to ensure that the samples sank to the bottom. The samples were polymerized at 65° C. for 48 hours in an oven and then left at room temperature for an additional 24 hours before sectioning. Sections of 70 to 80 nm thickness were obtained using a Leica UC/FC7 ultramicrotome (Leica Microsystems), deposited onto 300-mesh copper grids, and stained with 2% uranyl acetate/Reynolds lead citrate (Reynolds, 1963) for 1 minute. Grids were imaged using a JEOL 1200 EX TEM operating at 100 kV. Cell wall thickness was measured using ImageJ.

### Metabolic Activity Measurement Assay

Metabolic activity of the macrophages in the absence or in the presence of Mtb or inhibitor after 24 hours of incubation was determined using Deep Blue cell viability kits (cat #424702, BioLegend, San Diego, CA) following the manufacturer's instructions.

### Statistical Analysis

Statistical analyses were performed in Prism 9 (GraphPad, San Diego, CA). Statistical significance was defined as p<0.05.

### Results

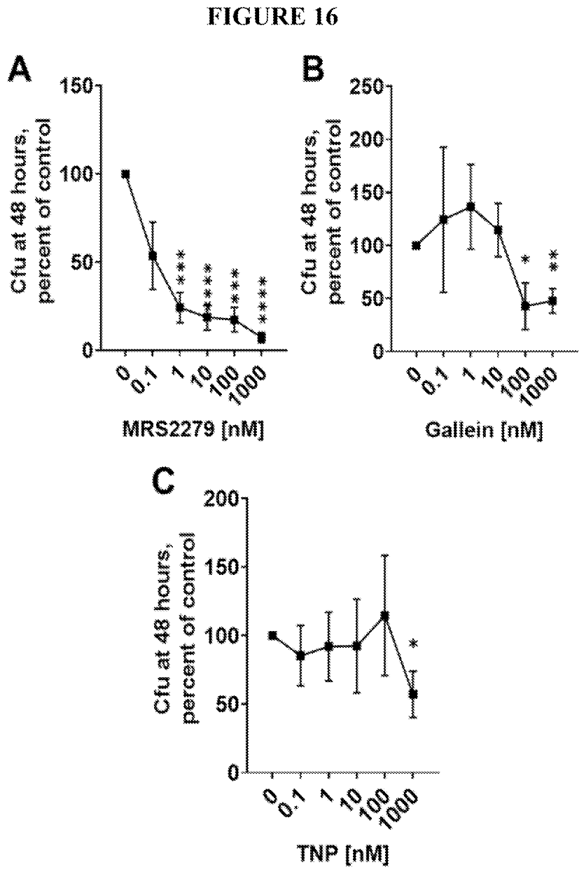

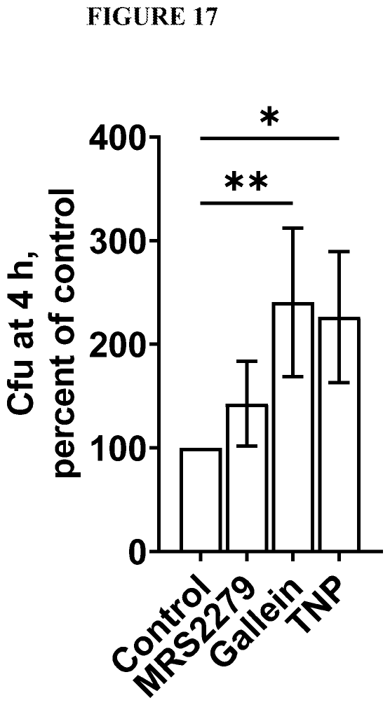

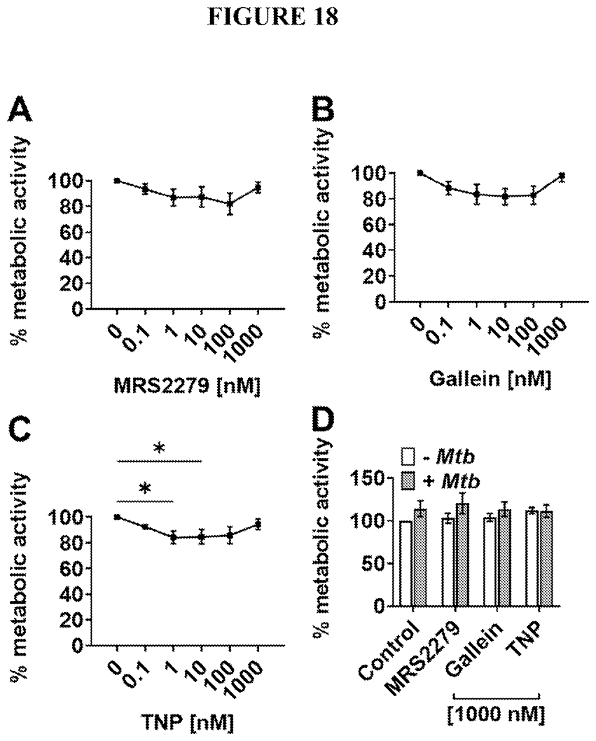

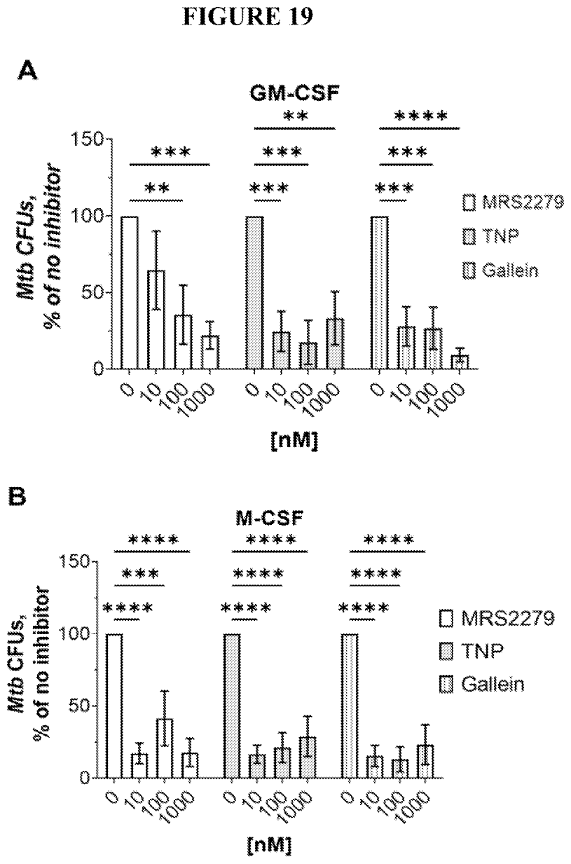

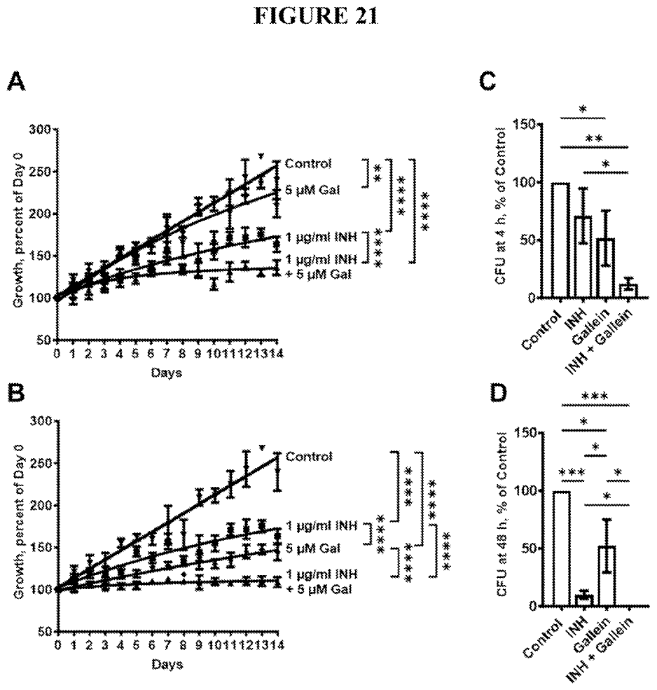

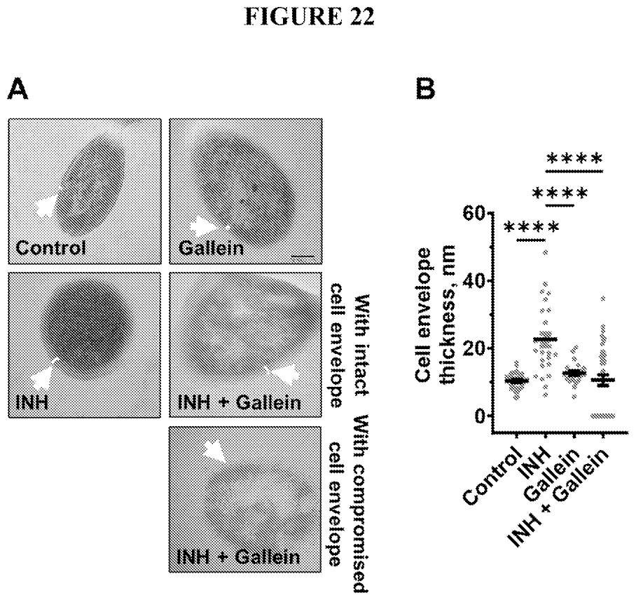

Compared to control, MRS2279 at concentrations at and above 1 nM reduced the viability of ingested Mtb in GM-CSF-generated macrophages at 48 hours of infection (FIG. 16A). Gallein at 100 and 1000 nM reduced the viability of ingested Mtb (FIG. 16B), and TNP at 1000 nM reduced the viability of Mtb by ~50% (FIG. 16C). To determine if the viability of ingested Mtb in GM-CSF macrophages treated with MRS2279, gallein, or TNP was reduced due to reduced ingestion of the Mtb, we tested the Mtb viability after 4 hours of infection in the presence or absence of 1000 nM inhibitor. Compared to control, MRS2279 did not significantly alter the number of ingested Mtb (FIG. 17), gallein and TNP increased the number of ingested Mtb after 4 hours of infection (FIG. 17). These results indicate that the observed effects of MRS2279, gallein, and TNP on numbers of viable Mtb in macrophages treated with GM-CSF at 48 hours (FIG. 16) are not due to the compounds causing the macrophages to ingest fewer Mtb. None of the concentrations of MRS2279 and gallein significantly altered the metabolic activity of macrophages treated with GM-CSF, and only 1 nM and 10 nM TNP reduced the metabolic activity of macrophages. (FIG. 18A-C). The inhibitors at 1000 nM did not significantly change the metabolic activity of macrophages infected with Mtb for 24 hours, suggesting that MRS 2279, gallein, and TNP reduce the viability of ingested Mtb without affecting the viability of macrophages (FIG. 18D). To test the effect of inhibitors on Mtb viability at a longer time of infection, inhibitors at 0, 10, 100, or 1000 nM were additionally added to the Mtb infected macrophages treated with either GM-CSF or M-CSF at 24 and at 48 hours of infection, and Mtb viability was tested at 72 hours of infection. Compared to control, 100 nM and higher MRS 2279, 10 nM and higher gallein, or 10 nM and higher TNP reduced the number of viable Mtb in GM-CSF macrophages (FIG. 19A). Compared to control, 10 nM and higher MRS2279, 10 nM and higher gallein, or 10 nM and higher TNP reduced the number of viable Mtb in M-CSF macrophages (FIG. 19B). Compared to control, MRS2279 at 100 and 1000 nM reduced the viability of ingested L. pneumophila in GM-CSF-generated macrophages at 48 hours of infection (FIG. 20A). TNP at 10, 100, and 1000 nM reduced the viability of ingested L. pneumophila (FIG. 20A), and gallein at 1000 nM reduced the viability of ingested L. pneumophila (FIG. 20A). Compared to control, MRS2279 at 10 and 1000 nM reduced the viability of ingested L. monocytogenes in GM-CSF-generated macrophages at 48 hours of infection (FIG. 20B). TNP at 10, 100, and 1000 nM reduced the viability of ingested L. monocytogenes (FIG. 20B), and gallein at 10, 100, and 1000 nM reduced the viability of ingested L. monocytogenes (FIG. 20B). Compared to control, 5 μM gallein slightly slowed growth (FIG. 21A), and 50 μM gallein inhibited growth by approximately 80% (FIG. 21B). In the absence of gallein, 1 μg/ml INH caused a partial but not complete reduction in Mtb growth (FIG. 21A and FIG. 21B), and this effect was potentiated by 5 and 50 μM gallein (FIG. 21A and FIG. 21B). Compared to control, gallein at 5 μM decreased ingested Mtb viability in GM-CSF-generated macrophages, and in the presence of 1 μg/ml INH significantly decreased the viability of ingested Mtb, with no detected surviving Mtb at 48 hours (FIG. 21C and FIG. 21D). As previously observed (Chuang et al. 2015), INH increased Mtb cell envelope thickness (FIG. 22A and FIG. 22B). 5 μM gallein alone did not significantly affect cell envelope thickness. For cells exposed to the combination of 1 μg/ml INH and 5 μM gallein, the average cell envelope thickness was comparable to control cells, but there were two distinct populations of Mtb cells (FIG. 22A and FIG. 22B). For 43.0±2.6% (mean±SEM, n=3) of the Mtb in the presence of INH and gallein, there was a detectable cell envelope, while the remaining Mtb had no detectable cell envelope (FIG. 22A and FIG. 22B).

### REFERENCES

Azevedo, C. and Saiardi, A. (2017). Eukaryotic Phosphate Homeostasis: The Inositol Pyrophosphate Perspective. Trends Biochem Sci 42, 219-231.
Bashatwah, R. M., Khanfar, M. A. and Bardaweel, S. K. (2018). Discovery of potent polyphosphate kinase 1 (PPK1) inhibitors using structure-based exploration of PPK1 Pharmacophoric space coupled with docking analyses. J Mol Recognit 31, e2726.
Boyer, J. L., Romero-Avila, T., Schachter, J. B. and Harden, T. K. (1996). Identification of competitive antagonists of the P2Y1 receptor. Mol Pharmacol 50, 1323-9.
Brennan, P. J. and Nikaido, H. (1995). The envelope of mycobacteria. Annu Rev Biochem 64, 29-63.
Cabou, C., Honorato, P., Briceno, L., Ghezali, L., Duparc, T., Leon, M., Combes, G., Frayssinhes, L., Fournel, A., Abot, A. et al. (2019). Pharmacological inhibition of the F(1)ATPase/P2Y(1) pathway suppresses the effect of apolipoprotein A1 on endothelial nitric oxide synthesis and vasorelaxation. Acta Physiol (Oxf) 226, e13268.
Chang, H., Yanachkov, I. B., Dix, E. J., Yanachkova, M., Li, Y., Barnard, M. R., Wright, G. E., Michelson, A. D. and Frelinger, A. L., 3rd. (2014). Antiplatelet activity, P2Y(1) and P2Y(1)(2) inhibition, and metabolism in plasma of stereoisomers of diadenosine 5′,5′″-P(1),P(4)-dithio-P(2),P(3)-chloromethylenetetraphosphate. PLOS One 9, e94780.
Chuang, Y. M., et al. (2015). Deficiency of the novel exopolyphosphatase Rv10216/PPX2 leads to metabolic downshift and altered cell wall permeability in Mycobacterium tuberculosis.mBio 6(2): e02428.
Cui, C., et al. (2021). Isolation of polymorphonuclear neutrophils and monocytes from a single sample of human peripheral blood. STAR Protoc 2(4): 100845.
Ghosh, S., Shukla, D., Suman, K., Lakshmi, B. J., Manorama, R., Kumar, S. and Bhandari, R. (2013). Inositol hexakisphosphate kinase 1 maintains hemostasis in mice by regulating platelet polyphosphate levels. Blood 122, 1478-86.
Gu, C., Stashko, M. A., Puhl-Rubio, A. C., Chakraborty, M., Chakraborty, A., Frye, S. V., Pearce, K. H., Wang, X., Shears, S. B. and Wang, H. (2019). Inhibition of Inositol Polyphosphate Kinases by Quercetin and Related Flavonoids: A Structure-Activity Analysis. J Med Chem 62, 1443-1454.
Huang, Z., Luo, Q., Guo, Y., Chen, J., Xiong, G., Peng, Y., Ye, J. and Li, J. (2015). Mycobacterium tuberculosis -Induced Polarization of Human Macrophage Orchestrates the Formation and Development of Tuberculous Granulomas In Vitro. PLOS One 10, e0129744.
Jeon, Y. T., Yang, W., Qiao, J. X., Li, L., Ruel, R., Thibeault, C., Hiebert, S., Wang, T. C., Wang, Y., Liu, Y. et al. (2014). Identification of 1-2-[4-chloro-1′-(2,2-dimethylpropyl)-7-hydroxy-1,2-dihydrospiro[indole-3,4′-piperidine]-1-yl]phenyl-3-5-chloro-[1,3]thiazolo[5,4-b]pyridin-2-ylurea, a potent, efficacious and orally bioavailable P2Y(1) antagonist as an antiplatelet agent. Bioorg Med Chem Lett 24, 1294-8.
Karim, Z. A., Vemana, H. P., Alshbool, F. Z., Lin, O. A., Alshehri, A. M., Javaherizadeh, P., Paez Espinosa, E. V. and Khasawneh, F. T. (2015). Characterization of a novel function-blocking antibody targeted against the platelet P2Y1 receptor. Arterioscler Thromb Vasc Biol 35, 637-44.
Lange, C., Dheda, K., Chesov, D., Mandalakas, A. M., Udwadia, Z. and Horsburgh, C. R., Jr. (2019). Management of drug-resistant tuberculosis. Lancet 394, 953-966.
Lawn, S. D. and Zumla, A. I. (2011). Tuberculosis. Lancet 378, 57-72.
Layhadi, J. A. and Fountain, S. J. (2019). ATP-Evoked Intracellular Ca(2+) Responses in M-CSF Differentiated Human Monocyte-Derived Macrophage are Mediated by P2X4 and P2Y11 Receptor Activation. Int J Mol Sci 20.
Lee, S., Kim, M. G., Ahn, H. and Kim, S. (2020). Inositol Pyrophosphates: Signaling Molecules with Pleiotropic Actions in Mammals. Molecules 25.
Lee, S., Park, B. B., Kwon, H., Kim, V., Jeon, J. S., Lee, R., Subedi, M., Lim, T., Ha, H., An, D. et al. (2022). TNP and its analogs: Modulation of IP6K and CYP3A4 inhibition. J Enzyme Inhib Med Chem 37, 269-279.
Lei, Y., Zhang, B., Liu, D., Zhao, J., Dai, X., Gao, J., Mao, Q., Feng, Y., Zhao, J., Lin, F. et al. (2020). Switching a Xanthine Oxidase Inhibitor to a Dual-Target Antagonist of P2Y(1) and P2Y(12) as an Oral Antiplatelet Agent with a Wider Therapeutic Window in Rats than Ticagrelor. J Med Chem 63, 15752-15772.
Liao, G., Ye, W., Heitmann, T., Ernst, G., DePasquale, M., Xu, L., Wormald, M., Hu, X., Ferrer, M., Harmel, R. K. et al. (2021). Identification of Small-Molecule Inhibitors of Human Inositol Hexakisphosphate Kinases by High-Throughput Screening. ACS Pharmacol Transl Sci 4, 780-789.
Lu, Y., Huang, J., Zhang, Y., Huang, Z., Yan, W., Zhou, T., Wang, Z., Liao, L., Cao, H. and Tan, B. (2021). Therapeutic Effects of Berberine Hydrochloride on Stress-Induced Diarrhea-Predominant Irritable Bowel Syndrome Rats by Inhibiting Neurotransmission in Colonic Smooth Muscle. Front Pharmacol 12, 596686.
Mane, N., Jimenez-Sabado, V. and Jimenez, M. (2016). BPTU, an allosteric antagonist of P2Y1 receptor, blocks nerve mediated inhibitory neuromuscular responses in the gastrointestinal tract of rodents. Neuropharmacology 110, 376-385.
Moritoh, Y., Abe, S. I., Akiyama, H., Kobayashi, A., Koyama, R., Hara, R., Kasai, S. and Watanabe, M. (2021). The enzymatic activity of inositol hexakisphosphate kinase controls circulating phosphate in mammals. Nat Commun 12, 4847.
Neville, N., Roberge, N., Ji, X., Stephen, P., Lu, J. L. and Jia, Z. (2021). A Dual-Specificity Inhibitor Targets Polyphosphate Kinase 1 and 2 Enzymes To Attenuate Virulence of Pseudomonas aeruginosa.mBio 12, e0059221.
Omotade, T. O. and Roy, C. R. (2019). Manipulation of Host Cell Organelles by Intracellular Pathogens. Microbiol Spectr 7.
Peng, J., Zhao, L., Wang, L., Chen, H., Qiu, Y., Wang, J., Yang, H., Liu, J. and Liu, H. (2018). Design, synthesis, and biological evaluation of 2-(phenoxyaryl)-3-urea derivatives as novel P2Y(1) receptor antagonists. Eur J Med Chem 158, 302-310.
Pi, Z., Sutton, J., Lloyd, J., Hua, J., Price, L., Wu, Q., Chang, M., Zheng, J., Rehfuss, R., Huang, C. S. et al. (2013). 2-Aminothiazole based P2Y(1) antagonists as novel antiplatelet agents. Bioorg Med Chem Lett 23, 4206-9.
Pilling, D., Vakil, V. and Gomer, R. H. (2009). Improved serum-free culture conditions for the differentiation of human and murine fibrocytes. J Immunol Methods 351, 62-70.
Puhl-Rubio, A. C., Stashko, M. A., Wang, H., Hardy, P. B., Tyagi, V., Li, B., Wang, X., Kireev, D., Jessen, H. J., Frye, S. V. et al. (2018). Use of Protein Kinase-Focused Compound Libraries for the Discovery of New Inositol Phosphate Kinase Inhibitors. SLAS Discov 23, 982-988.
Rajasekaran, S. S., Illies, C., Shears, S. B., Wang, H., Ayala, T. S., Martins, J. O., Dare, E., Berggren, P. O. and Barker, C. J. (2018). Protein kinase-and lipase inhibitors of inositide metabolism deplete IP(7) indirectly in pancreatic beta-cells: Off-target effects on cellular bioenergetics and direct effects on IP6K activity. Cell Signal 42, 127-133.
Reynolds, E. S. (1963). The use of lead citrate at high pH as an electron-opaque stain in electron microscopy. J Cell Biol 17 (1), 208-212.
Rijal, R., Cadena, L. A., Smith, M. R., Carr, J. F. and Gomer, R. H. (2020). Polyphosphate is an extracellular signal that can facilitate bacterial survival in eukaryotic cells. Proc Natl Acad Sci U S A 117, 31923-31934.
Rock, J. M., Hopkins, F. F., Chavez, A., Diallo, M., Chase, M. R., Gerrick, E. R., Pritchard, J. R., Church, G. M., Rubin, E. J., Sassetti, C. M. et al. (2017). Programmable transcriptional repression in mycobacteria using an orthogonal CRISPR interference platform. Nat Microbiol 2, 16274.
Ruel, R., L'Heureux, A., Thibeault, C., Lapointe, P., Martel, A., Qiao, J. X., Hua, J., Price, L. A., Wu, Q., Chang, M. et al. (2013). Potent P2Y1 urea antagonists bearing various cyclic amine scaffolds. Bioorg Med Chem Lett 23, 6825-8.
Russell, D. G., Mwandumba, H. C. and Rhoades, E. E. (2002). Mycobacterium and the coat of many lipids. J Cell Biol 158, 421-6.
Samah, S., Fatah, C., Jean-Marc, B., Safia, K. T. and Fatima, L. D. (2017). Purification and characterization of Cc-Lec, C-type lactose-binding lectin: A platelet aggregation and blood-clotting inhibitor from Cerastes cerastes venom. Int J Biol Macromol 102, 336-350.
Sampson, S. L., Dascher, C. C., Sambandamurthy, V. K., Russell, R. G., Jacobs, W. R., Jr., Bloom, B. R. and Hondalus, M. K. (2004). Protection elicited by a double leucine and pantothenate auxotroph of Mycobacterium tuberculosis in guinea pigs. Infect Immun 72, 3031-7.
Shears, S. B. (2018). Intimate connections: Inositol pyrophosphates at the interface of metabolic regulation and cell signaling. J Cell Physiol 233, 1897-1912.
Singh, M., Tiwari, P., Arora, G., Agarwal, S., Kidwai, S. and Singh, R. (2016). Establishing Virulence Associated Polyphosphate Kinase 2 as a drug target for Mycobacterium tuberculosis. Sci Rep 6, 26900.
Slauch, J. M. (2011). How does the oxidative burst of macrophages kill bacteria? Still an open question. Mol Microbiol 80, 580-3.
Sturgill-Koszycki, S., Schlesinger, P. H., Chakraborty, P., Haddix, P. L., Collins, H. L., Fok, A. K., Allen, R. D., Gluck, S. L., Heuser, J. and Russell, D. G. (1994). Lack of acidification in Mycobacterium phagosomes produced by exclusion of the vesicular proton-ATPase. Science 263, 678-81.
Tantilertanant, Y., Niyompanich, J., Everts, V., Supaphol, P., Pavasant, P. and Sanchavanakit, N. (2019). Cyclic tensile force stimulates BMP9 synthesis and in vitro mineralization by human periodontal ligament cells. J Cell Physiol 234, 4528-4539.
Watala, C., Wzorek, J., Palma, A. and Boncler, M. (2022). A comparison of different regression models for the quantitative analysis of the combined effect of P2Y(12) and P2Y(1) receptor antagonists on ADP-induced platelet activation. Thromb Res 211, 88-97.
Wormald, M. M., Ernst, G., Wei, H. and Barrow, J. C. (2019). Synthesis and characterization of novel isoform-selective IP6K1 inhibitors. Bioorg Med Chem Lett 29, 126628.
Yanachkov, I. B., Chang, H., Yanachkova, M. I., Dix, E. J., Berny-Lang, M. A., Gremmel, T., Michelson, A. D., Wright, G. E. and Frelinger, A. L., 3rd. (2016). New highly active antiplatelet agents with dual specificity for platelet P2Y1 and P2Y12 adenosine diphosphate receptors. Eur J Med Chem 107, 204-18.
Yang, H., Xu, S., Li, J., Wang, L. and Wang, X. (2015). Potassium 2-(1-hydroxypentyl)-benzoate inhibits ADP-induced rat platelet aggregation through P2Y1-PLC signaling pathways. Naunyn Schmiedebergs Arch Pharmacol 388, 983-90.
Yang, W., Wang, Y., Lai, A., Qiao, J. X., Wang, T. C., Hua, J., Price, L. A., Shen, H., Chen, X. Q., Wong, P. et al. (2014). Discovery of 4-aryl-7-hydroxyindoline-based P2Y1 antagonists as novel antiplatelet agents. J Med Chem 57, 6150-64.
Zhou, Y., Mukherjee, S., Huang, D., Chakraborty, M., Gu, C., Zong, G., Stashko, M. A., Pearce, K. H., Shears, S. B., Chakraborty, A. et al. (2022). Development of Novel IP6K Inhibitors for the Treatment of Obesity and Obesity-Induced Metabolic Dysfunctions. J Med Chem 65, 6869-6887.

WO 2018/192051A1 to Terao et al. IP6k inhibitors.

1. A method of treating a bacterial infection in a subject comprising administering to the subject a therapeutically effective amount of a P2Y1 inhibitor.

2. The method of claim 1 wherein the bacterial infection is an infection of Mycobacterium tuberculosis, Pseudomonas aeruginosa, Legionella pneumophila, or Listeria monocytogenes.

3. The method of claim 1 wherein the subject is a human, a non-human primate, a bovine, an equine, a porcine, a canine, a feline, a guinea pig, a rat, a hamster, a rabbit, a mouse, or a mole.

4. The method of claim 1 wherein the subject is a human.

5. The method of claim 1 wherein the P2Y1 inhibitor is: MRS2279, MRS2179, MRS2500, BPTU, a 4-aryl-7-hydroxylindoline derivative, diadenosine polyphosphate, suramin, a 2(phenoxyaryl)-3-urea derivative, Cc-lec, AP4A analogs, potassium 2-(1-hydroxypentyl)-benzoate, EL2Ab, Compound 10q, Compound 20c (BMS-884775), Compound 4a, Compound 7j, or A3P5PS.

6. The method of claim 1 wherein the P2Y1 inhibitor is MRS2279.

7. A method of treating a bacterial infection in a subject comprising administering to the subject a therapeutically effective amount of a PPK1 or PPK2 inhibitor.

8. The method of claim 7 wherein the bacterial infection is an infection of Mycobacterium tuberculosis, Pseudomonas aeruginosa, Legionella pneumophila, or Listeria monocytogenes.

9. The method of claim 7 wherein the subject is a human, a non-human primate, a bovine, an equine, a porcine, a canine, a feline, a guinea pig, a rat, a hamster, a rabbit, a mouse, or a mole.

10. The method of claim 7 wherein the subject is a human.

11. The method of claim 7 wherein the PPK1 or PPK2 inhibitor is: NSC35676, NSC30205, NSC345647, NSC9037, Inh1, Inh2, or gallein.

12. The method of claim 7 wherein the PPK1 or PPK2 inhibitor is gallein.

13. A method of treating a bacterial infection in a subject comprising administering to the subject a therapeutically effective amount of an IP6K inhibitor.

14. The method of claim 13 wherein the bacterial infection is an infection of Mycobacterium tuberculosis, Pseudomonas aeruginosa, Legionella pneumophila, or Listeria monocytogenes.

15. The method of claim 13 wherein the subject is a human, a non-human primate, a bovine, an equine, a porcine, a canine, a feline, a guinea pig, a rat, a hamster, a rabbit, a mouse, or a mole.

16. The method of claim 13 wherein the subject is a human.

17. The method of claim 13 wherein the IP6K inhibitor is a thiadiazolidinone compound.

18. The method of claim 13 wherein the IP6K inhibitor is TNP, a TNP analog, Compound 9, Compound 20 (UNC7467), SC-919, Compound 24, LY 294002, PAO, U73122, LI-1753, LI-1851, LI-2355, LI-2356, LI-2386, LI-2124, LI-2172, LI-2240, LI-2260, LI-2180, LI-2178, LI-2242, LI-2263, LI-2406, UNC10102221, UNC10104261, UNC10105760, UNC10225357, or diosmetin.

19. The method of claim 13 wherein the IP6K inhibitor is TNP.

20. A method of treating a bacterial infection in a subject comprising administering concomitantly a therapeutically effective amount to the subject of two or more of a P2Y1 inhibitor, a PPK1 or PPK2 inhibitor, or an IP6K inhibitor.
